(19) United States
(12) Patent Application Publication
(10) Pub. No.: US 2024/0285810 A1
(43) Pub. Date: Aug. 29, 2024

(54) DEVELOPMENT OF A NOVEL PET PROBE FOR TARGETED BACTERIAL IMAGING

(71) Applicant: UNIVERSITY OF CINCINNATI, Cincinnati, OH (US)

(72) Inventors: Nalinikanth KOTAGIRI, West Chester, OH (US); Nabil SIDDIQUI, Cincinnati, OH (US)

(21) Appl. No.: 17/912,167

(22) PCT Filed: Jun. 26, 2020
(86) PCT No.: PCT/US2020/039839

§ 371 Date: Sep. 16, 2022

Related U.S. Application Data
(60) Provisional application No. 63/025,056, filed on May 14, 2020.
(60) Provisional application No. 62/868,408, filed on Jun. 28, 2019.

(51) Int. Cl. A61K 51/04

(52) U.S. Cl. CPC A61K 51/048; A61K 51/0453

(57) ABSTRACT

A probe for positron emission tomography (PET) is disclosed. The probe is selected from the group consisting of yersini-abactin (Ybt) labeled with Copper-64; staphylopine (StP) labeled with Copper-64; yersiniabactin (Ybt) labeled with Zirconium-89; and staphylopine (StP) labeled with Zirconium-89.

### CROSS REFERENCE TO RELATED APPLICATIONS

This application claims priority to U.S. Provisional Application Ser. No. 62/868,408, filed Jun. 28, 2019, and U.S. Provisional Application Ser. No. 63/025,056, filed May 14, 2020, which applications are hereby incorporated by reference in their entirety.

### TECHNICAL FIELD

The present invention relates to positron emission tomography probes.

### BACKGROUND OF THE INVENTION

Despite significant medical advances, selective visualization of bacterial species in vivo remains a big challenge. Standard diagnostic techniques, such as microbiological culture or molecular identification, can be fast and reliable with easily obtained samples such as blood or urine. However, in cases of deep-seated bacterial colonization, biopsies are often required, which can be highly invasive, costly and susceptible to errors. Conventional radiological techniques such as computerized tomography (CT), magnetic resonance imaging (MRI), positron emission tomography (PET), X-rays, and ultrasound are also employed. However, they are unable to differentiate bacterial infections from sterile inflammation or cancer.

Positron emission tomography (PET) is a medical imaging technique that is commonly used to produce three-dimensional images in vivo. This technique detects gamma rays emitted by a positron-emitting radionuclide tracer that has been administered to a subject. Three-dimensional images of tracer concentrations within the subject's body can then be constructed by computational analysis. In this way, PET can be used, for example, to observe a variety of organs and/or physiological processes such as cellular metabolism and protein synthesis, as well as pathological conditions such as cancer and heart disease. An example of a common clinical PET probe is ¹⁸F-Fluoro-2-Deoxy-D-glucose (¹⁸F-FDG). It is used, for example, to diagnose certain pathological conditions including cancer as well as to evaluate the effects of certain therapeutic regimens. However, a need still exists for a means to observe bacterial infections in vivo using PET probes.

### SUMMARY OF THE INVENTION

The invention disclosed herein provides compounds that are useful as probes in processes such as positron emission tomography, as well as methods for making and using them. The invention disclosed herein has a number of embodiments. One embodiment of the invention is a composition of matter comprising a positron emission tomography probe selected from the group consisting of yersiniabactin (Ybt) labeled with Copper-64; staphylopine (StP) labeled with Copper-64; yersiniabactin (Ybt) labeled with Zirconium-89; and staphylopine (StP) labeled with Zirconium-89. In one embodiment, the PET probe is yersiniabactin (Ybt) labeled with Copper-64. In another embodiment, the PET probe is staphylopine (StP) labeled with Copper-64. In another embodiment, the PET probe is yersiniabactin (Ybt) labeled with Zirconium-89. In another embodiment, the PET probe is staphylopine (StP) labeled with Zirconium-89.

Embodiments of the invention include methods for using the PET probes to identify a bacterial infection in vivo in a mammal. In one embodiment, the method comprises the steps of: (a) administering a composition to the mammal comprising a positron emission tomography (PET) probe selected from the group consisting of yersiniabactin (Ybt) labeled with Copper-64, staphylopine (StP) labeled with Copper-64, yersiniabactin (Ybt) labeled with Zirconium-89, and staphylopine (StP) labeled with Zirconium-89; (b) allowing the probe to accumulate in the mammal; and (c) observing the accumulated probe in the mammal using a positron emission tomography process; so that a concentration of the probe in the mammal is observed.

In another embodiment, the bacterial infection that is observed in vivo comprises bacteria selected from the group consisting of E. coli UTI89 (Gram-negative) and K. pneumoniae. In one embodiment, the bacterial infection that is observed in vivo comprises E. coli UTI89 (Gram-negative) bacteria. In another embodiment, the bacterial infection that is observed in vivo comprises K. pneumoniae bacteria.

In one embodiment of the present invention, the method for using the PET probes to identify a bacterial infection in vivo in a mammal comprises a positron emission tomography (PET) probe that is yersiniabactin (Ybt) labeled with Copper-64. In another embodiment, the method for using the PET probes to identify a bacterial infection in vivo in a mammal comprises a positron emission tomography (PET) probe that is yersiniabactin (Ybt) labeled with Zirconium-89.

In one embodiment of the present invention, the concentration of the probe is used to detect a urinary tract infection. In another embodiment, the concentration of the probe is used to monitor treatment response, progression, or both in antibiotic resistant E. coli. In another embodiment, the positron emission tomography probe is administered to the mammal in combination with a pharmaceutically acceptable compound comprising a diluent, a carrier, or a binding agent.

Embodiments of the invention include methods for using the PET probes to selectively observe therapeutic bacteria in vivo in a mammal. In one embodiment, the method comprises the steps of: (a) administering a composition to the mammal comprising therapeutic bacteria; (b) allowing the therapeutic bacteria to selectively accumulate in the mammal; (c) administering a composition to the mammal comprising a positron emission tomography (PET) probe selected from the group consisting of: yersiniabactin (Ybt) labeled with Copper-64, staphylopine (StP) labeled with Copper-64, yersiniabactin (Ybt) labeled with Zirconium-89, and staphylopine (StP) labeled with Zirconium-89; (d) allowing the probe to selectively accumulate in the mammal; and (e) observing the accumulated probe in the mammal using a positron emission tomography; so that the therapeutic bacteria is selectively observed in vivo in the mammal.

In one embodiment, the method to selectively observe therapeutic bacteria in vivo in a mammal uses therapeutic bacteria that is a non-pathogenic strain of E. coli. In another embodiment, the positron emission tomography probe is administered to the mammal in combination with a pharmaceutically acceptable compound comprising a diluent, a carrier, or a binding agent. In one embodiment, the method to selectively observe therapeutic bacteria in vivo in a mammal uses a positron emission tomography (PET) probe that is yersiniabactin (Ybt) labeled with Copper-64. In another embodiment, the method to selectively observe therapeutic bacteria in vivo in a mammal uses a positron emission tomography (PET) probe that is yersiniabactin (Ybt) labeled with Zirconium-89.

Other embodiments of the invention include kits, for example, those including a plurality of containers that hold one or more reagents useful in a PET process. In one illustrative embodiment, the kit includes one or more compounds selected from the group consisting of yersiniabactin (Ybt) labeled with Copper-64; staphylopine (StP) labeled with Copper-64; yersiniabactin (Ybt) labeled with Zirconium-89; and staphylopine (StP) labeled with Zirconium-89. In some embodiments of the invention, the kit includes articles useful to administer the probe, for example a capsule that can surround the probe (e.g. for use when the probe is administered orally) or a needle and syringe (e.g. for use when the probe is administered parenterally).

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1A is a graph showing Ex vivo biodistribution of ⁶⁴Cu-Ybt 2 hr post injection (n=1). This figure shows myositis infection.

FIG. 1B is a graph showing Ex vivo biodistribution of ⁶⁴Cu-Ybt 24 hr post injection (n=3). This figure shows myositis infection.

FIG. 1C is a graph showing Ex vivo biodistribution of ¹⁸F-FDG 1 hr post injection (n=1). This figure shows myositis infection.

FIG. 1D is a graph showing Ex vivo biodistribution of ⁶⁴Cu-Ybt 24 hr post injection (n=1). This figure shows tumor localization of bacteria.

FIG. 2A is a drawing of the structure of staphylopine (StP). MW=328.33 and log P=−2.47.

FIG. 2B is a drawing of the structure of yersiniabactin (YbT). MW=481.64 and log P=3.74.

FIG. 2C is a drawing of radiolabeled metallophores as targeted PET probes.

FIG. 2D is an illustration of radiolabeled metallophores.

FIG. 3A is shows [M+H]⁺ peaks for the standard and natural StP molecule synthesized and secreted by S. aureus.

FIG. 3B shows [M+H]⁺ peaks for the two isomers of YbT molecule by E. coli UTI89 obtained from LC-MS analyses.

FIG. 4 shows that StP and YbT has the highest affinity for ⁸⁹Zr. StP and YbT complexations with Cobalt-55, Gallium-68 and Zirconium-89 are shown in various physicochemical conditions

FIG. 5A shows the percentage of ⁸⁹Zr labeled YbT in saline and in DTPA-supplemented saline over 24 hrs.

FIG. 5B shows the percentage of ⁸⁹Zr labeled StP in saline and in DTPA-supplemented saline over 24 hrs.

FIG. 6 is a schematic showing the targeted imaging of pathogenic bacteria.

FIG. 7 shows ex vivo biodistribution of ⁶⁴Cu-YbT 24 hrs post-injection in mice (n=3, results are shown as mean±s.d.).

FIG. 8 shows static PET/CT imaging of Ex vivo biodistribution of ⁶⁴Cu-YbT 24 hrs post-injection in mice (n=1).

FIG. 9 shows Ex vivo biodistribution of ¹⁸F-FDG 2 hrs post-injection in mice (n=1).

FIG. 10 shows Ex vivo biodistribution of ⁶⁴Cu-YbT 24 hrs post-injection in mice (n=2, results are shown as mean±s.d.).

FIGS. 11A, 11B and 11C are tables showing the results of the radiolabeling procedure.

FIG. 11D shows the results of a different iteration of the radiolabeling procedure with ⁸⁹Zr.

FIG. 12 is a graph showing the biodistribution (performed at 1 h) of yersiniabactin at 1 h by % ID/g.

FIG. 13 is a graph showing the biodistribution (performed at 4 h) of yersiniabactin at 4 h by % ID/Organ.

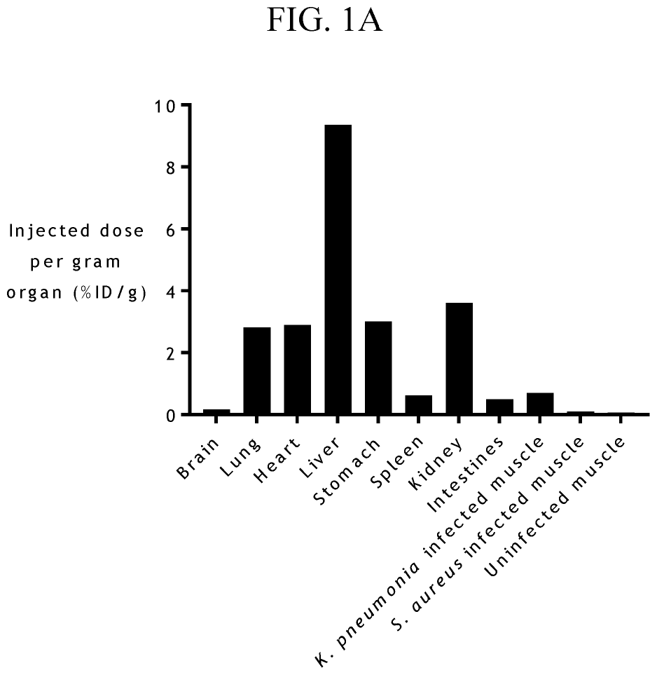

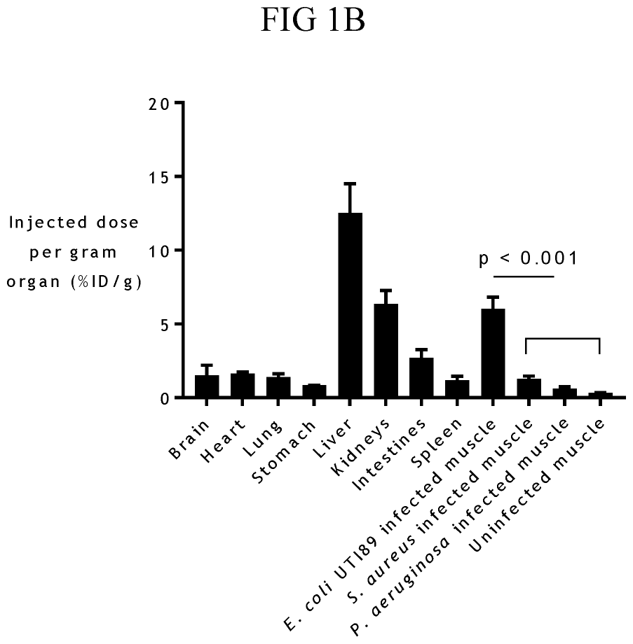

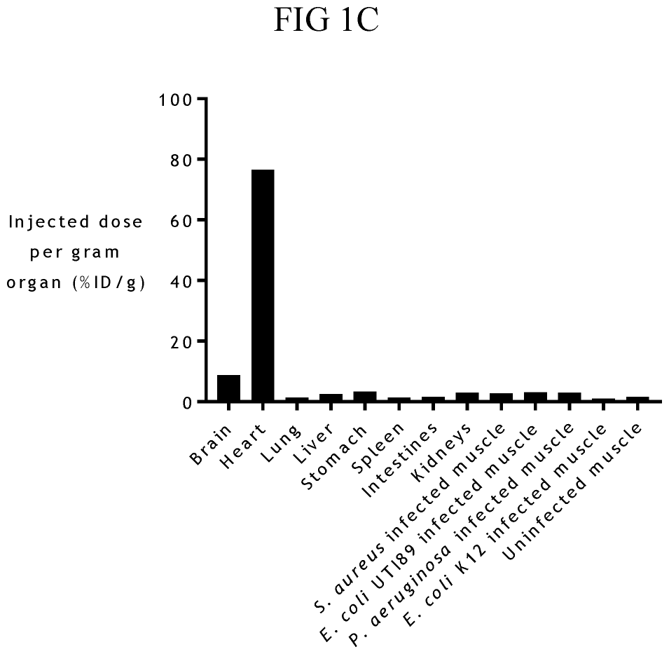

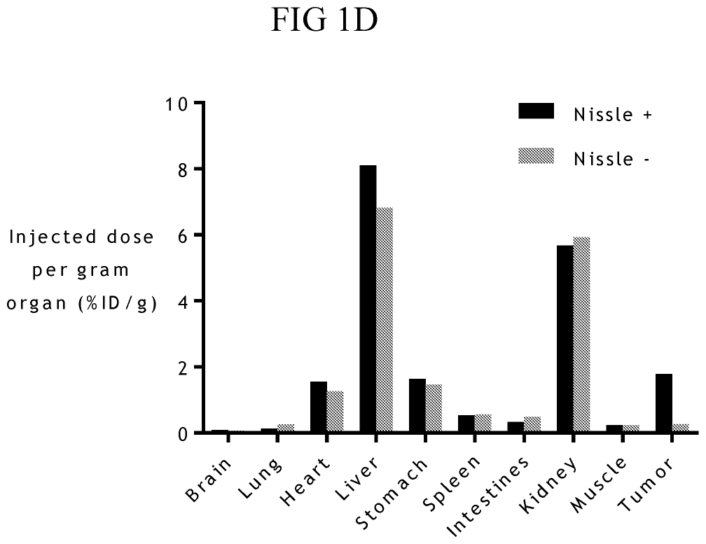

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

From a medical standpoint, precise identification of bacterial presence in the human body is imperative. Pathogenic species need to be selectively located during the initial stages of an infection for the initiation of appropriate medical and surgical management, thus preventing antibiotic abuse and development of multi-drug resistance bacteria. Equally important is the accurate mapping of tumor-targeting bacteria and bacterial vesicles for cancer therapy, not only for confirming the successful localization in the tumor to assess the therapeutic effect, but also for monitoring biodistribution in other organs to predict off-target toxicity.

Numerous molecular imaging techniques such as ultrasound (US), computed tomography (CT), magnetic resonance imaging (MRI), single-photon emission CT (SPECT), and positron emission tomography (PET) have been developed for preclinical and clinical research in the last three decades. However, current clinical probes cannot reliably distinguish bacteria from mammalian cells in vivo. A comprehensive understanding of bacterial biology is required to develop probes for targeted imaging. Bacteria are evolutionarily and phylogenetically distinct from mammalian cells; fundamental differences in metabolism and cellular structures can be leveraged to develop bacteria-specific imaging agents.

Metal transport is a pathway in bacteria that can be exploited to develop specific probes. Metallophores (Mtps) are small peptide-like molecules that possess a high affinity for transition metals. Even though Mtps have been studied since the 1970s for their role in metal homeostasis in bacteria and fungi, research has largely been limited to modifying these biomolecules into novel antibacterials. In the diagnostic field, Mtp has received attention as a potential tool for molecular imaging for fungal infections only recently. In Aspergillus fumigatus, MirB is a specific protein that regulates the transport of the metallophore, triacetylfusarinine C (TAFC). Since mammalian cells do not possess MirB, gallium-68 labeled TAFC has been shown to be an excellent PET probe in delineating the fungal pathogen in A. fumigatus rat infection models.

One embodiment of the present invention is a novel PET probe to selectively image bacteria in vivo. The metal-chelating compounds, yersiniabactin (Ybt) and staphylopine (StP), are labeled with clinically used PET radionuclides, Copper-64 (t_{1/2}=12.7 hrs) and Zirconium-89 for targeted imaging of bacteria inside the body. The present invention has surprisingly found that, unlike the clinically used ¹⁸F-FDG, the probes of the present invention (⁶⁴Cu-Ybt, ⁸⁹Zr-YbT, ⁶⁴Cu-StP and ⁸⁹Zr-StP) can be either used as highly selective bacterial PET probes or differentiate bacterial species from one another for multiple biomedical applications. The present invention is highly specific to certain pathogenic strains of E. coli. It can be used to detect non-pathogenic strains of E. coli that are typically used as probiotics and for other bioengineering applications. In addition, the present invention can be used to detect urinary tract infections, nosocomial pneumonia, and infections related to pathogenic E. coli and K. pneumonia in the clinics. It can be used to monitor treatment response and progression in antibiotic resistant E. coli. It can also be used to track bioengineered/probiotic E. coli in the body. Those skilled in the art of microbiome research will recognize the variety of uses of this highly selective contrast agent.

The present invention enables the imaging of bacterial localization in at least two very different in vivo settings. Intramuscular myositis infection models involving E. coli UTI89, E. coli K12, K. pneumonia, P. aeruginosa and S. aureus were developed. In addition, tumor models were developed via subcutaneous injection of 4T1 cells, followed by tail-vein injection of E. coli Nissle to study localization of bacteria in tumors (see Example 1).

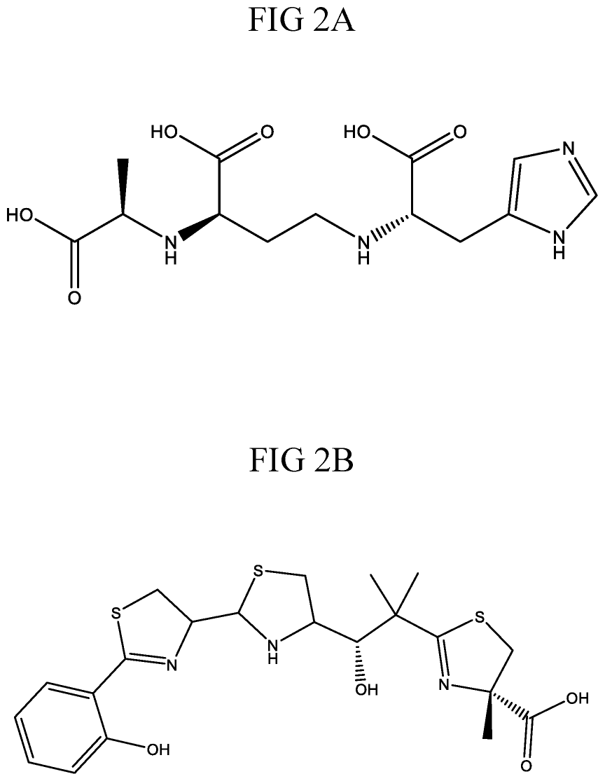

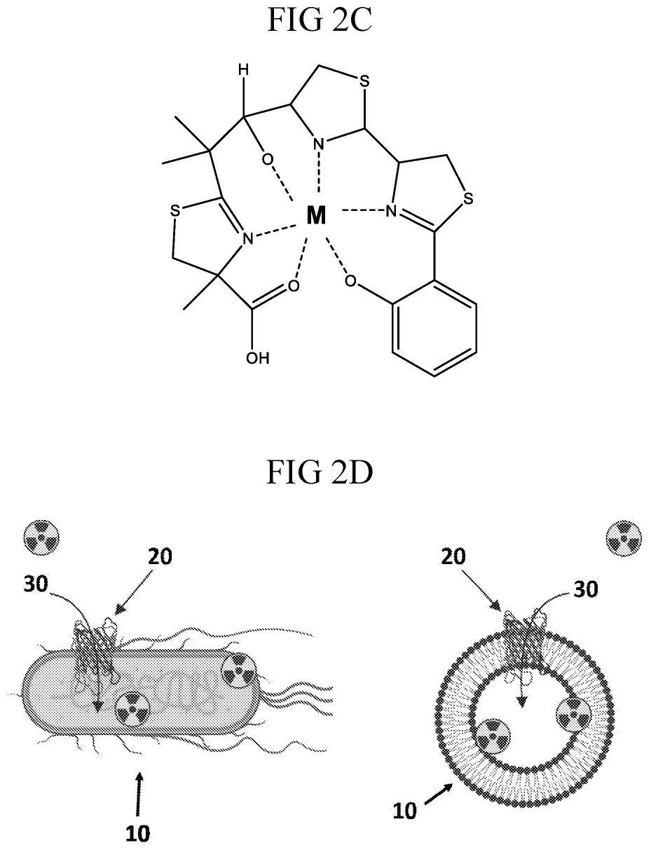

Recent research on copper homeostasis in E. coli and S. aureus underscores the roles of transport proteins FyuA and CntA respectively. Pathogenic E. coli UTI89 uses the metallophore yersiniabactin (YbT) to sequester Cu (II) from the extracellular environment inside the bacteria. Metal bound Ybt is first selectively “recognized” by the outer membrane protein FyuA (ferric yersiniabactin uptake A), before the inner membrane ATP-binding cassette transporters, YbtPQ allow cytoplasmic entry. In S. aureus the metallophore, Staphylopine (StP), chelates metals and is selectively “captured” by the CntA domain before being transferred inside the bacteria by the CntBCDF domains of this ABC transporter protein. In the present invention, we have found that these bacterial metal transporters can be targeted by clinically relevant radioisotope labeled metallophores (FIGS. 2A-D). Referring to FIG. 2D, a bacteria (10) with a FyuA receptor (20) is radiolabeled with M-YbT (30). The “M” in M-YbT indicates a radiometal (examples include ⁶⁴Cu or ⁸⁹Zr).

Even though numerous bacteria-specific PET probes have been developed in the last decade, most of these involve complex and time-consuming reaction mechanisms that eventually yield in the synthesis of the tracer. Furthermore, most tracers do not have high complexation (>90%) with the radioisotope, which necessitates an additional purification step before administration. These might prove as potential barriers to clinical translation as radiologists in hospitals would ideally prefer to be able to prepare the probes in a facile manner. In some of the initial preclinical and clinical investigations, radiolabeled antibiotics such as ^{99m}Tc-ciprofloxacin seemed promising, not only for their ability to specifically kill (or disable) bacteria while being nontoxic to human cells, but also for the ease of probe preparation using manufacturable kits. However, in later investigations these SPECT probes proved to not only accumulate in bacterial lesions, but also in sterile inflammatory sites.

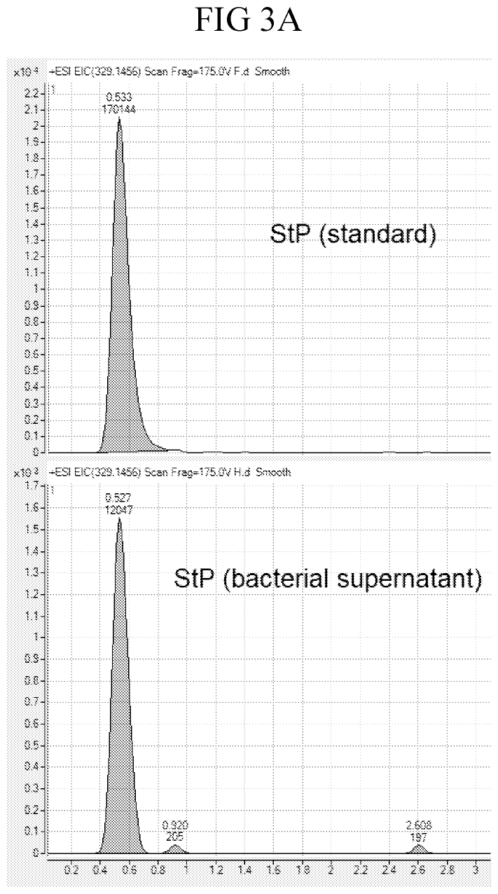

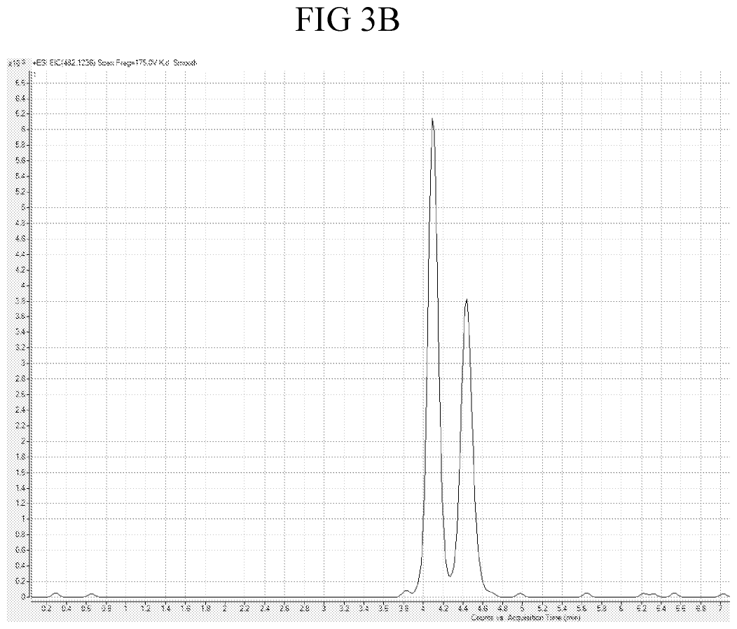

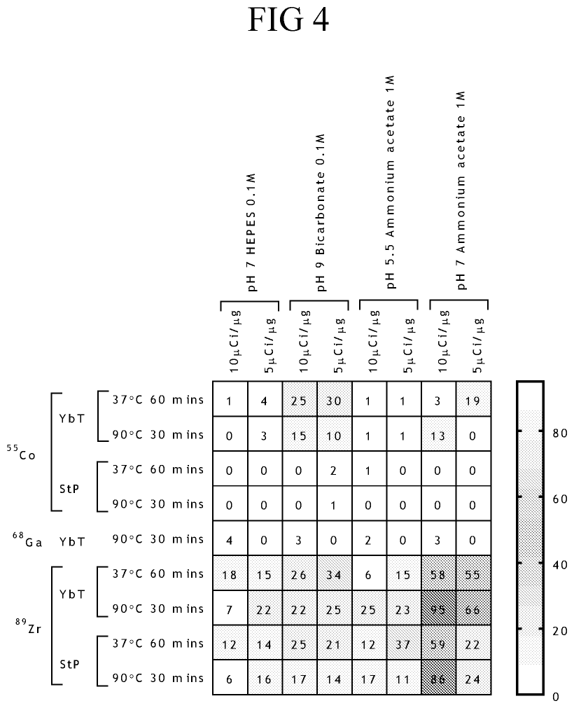

Metallophore-based probes eliminate the need for sophisticated synthetic chemical reactions since these molecules are readily synthesized and secreted by bacteria and can easily be obtained using simple purification strategies from the culture supernatant (FIGS. 3A-B). In the present invention, we have found that ⁶⁴Cu and ⁸⁹Zr-labeled StP and YbT are particularly useful probes when used to understand uptake in selective species of bacteria. For example, when we performed in vitro radiolabeling of StP and YbT with three different PET-emitting radiometals, we observed the highest complexations of StP and YbT with Zirconium-89 (FIG. 4).

⁶⁴Cu has a half-life (t_{1/2}) of 12.7 hrs, which allows short-term imaging of up to 48 hrs post-administration of the probe, and radioactive clearance from the body in less than a week. ⁸⁹Zr with its t_{1/2} of 3.3 d has the advantage of allowing precise longitudinal imaging 8 days following a single administration of the probe. However, the significantly longer t1/2 means that radioactivity can remain in the body for up to a month. This is of particular concern with ⁸⁹Zr-based probes as preclinical studies involving this radiometal often report significant bone accumulation, resulting from dissociation of the metal from the tracer. However, as shown in Example 2, the radiometal in ⁸⁹Zr labeled StP dissociated significantly from StP within the first hour. ⁸⁹Zr-labeled YbT probes accumulated more significantly in the liver than anywhere else in the body, which confirms in vivo stability.

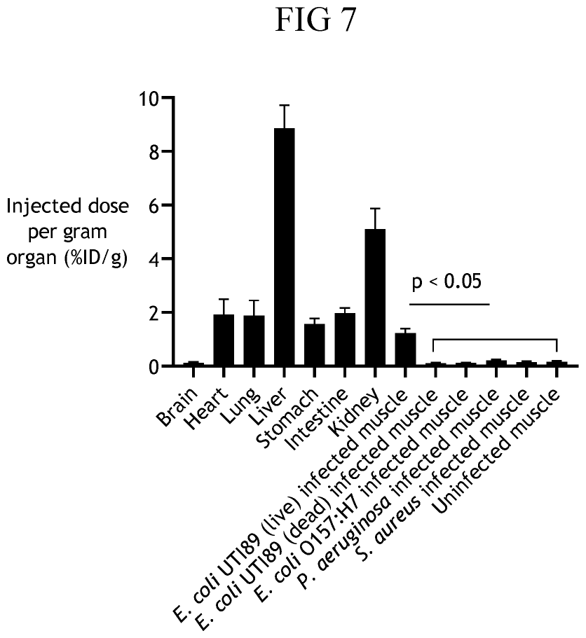

Since ⁶⁴Cu and ⁸⁹Zr-labeled YbT proved to the best amongst all the probes investigated thus far, we wanted to examine whether the YbT probes could selectively differentiate its target, E. coli UTI89 (Gram-negative), from representative Gram-negative and Gram-positive bacteria that lack the FyuA receptor in vivo. As discussed in Example 3, tail-vein injections of ⁶⁴Cu-YbT in myositis infection models revealed significantly higher accumulation of the probe in UTI89 infected thigh muscles than in S. aureus or P. aeruginosa infected muscles. While these are encouraging data, one could argue that E. coli may possess mechanisms to import the radiometal labeled metallophore other than the FyuA-mediated uptake. Consequently, we decided to compare the localization of ⁶⁴Cu-YbT in another highly pathogenic strain of E. coli, O157:H7, which does not encode for the FyuA receptor. Intravenous administration of the probe revealed PET signals similar to those observed before, thus confirming the strain-specific imaging ability of the probe. It was important for us to also ensure that our probe was taken up by live UTI89 and not by the dead bacteria, so we confirmed our hypothesis by following the same experimental approach as we did for other bacteria. Ex vivo biodistribution studies revealed concordant results to all the PET/CT images (FIG. 7).

### Bacteria-Based Cancer Imaging

The present invention can use the disclosed probes to track therapeutic bacteria in vivo. The specificity of these distinct metal transporters for their corresponding metal bound Mtps can be leveraged for in vivo visualization of therapeutic bacteria. For example, engineered probiotic commensal E. coli Nissle can be used to target metastatic lymphoma. Nissle is a commensal strain of E. coli that traffics FyuA-mediated metal-bound YbT. We have found that the probes of the present invention can selectively determine Nissle localization in tumors in vivo. Results are shown in Example 6.

Advances in bioengineering have enabled us to explore not only bacteria, but also diverse bacterial vesicles for their anti-tumor activity. Bacterial outer membrane vesicles (OMVs) are nano-sized acellular structures that are naturally secreted by all Gram-negative bacteria. Amongst all the cell-derived nanovesicular systems that have been investigated in the last decade, OMVs have proved to possess the most superior characteristics owing to their high biostability and lack of immunogenicity. Furthermore, most of these extracellular vesicles have been generated from E. coli which means that genetic modifications of the bacteria can yield large quantities of customized vesicles that can be easily obtained via optimized purification procedures. We have found that OMVs secreted by E. coli Nissle can be selectively tracked by radiolabeled YbT without the need for further genetic manipulation of the bacteria. We incubated Nissle OMVs and milk exosomes with ⁶⁴Cu-YbT, and subsequently injected both radiolabeled nanovesicles in tumor-bearing mice. PET/CT imaging revealed radioactive concentration in tumor of the mouse injected with OMVs, but no signal from that in exosome administered mouse. FyuA-expressing E. coli species have been proven to naturally possess the metal transporter in their secreted OMVs. Therefore, without being bound by theory, we think that the presence of FyuA on the lipid bilayer allowed selective entry and retention of ⁶⁴Cu-YbT inside the OMVs, unlike the exosomes that lack the metal transporter.

Current imaging tools that are clinically available lack selectivity for bacteria; they only measure downstream consequences and therefore, cannot reliably distinguish infections from other diseases, such as diabetes, cancer, and immunosuppression that elicit similar physiological responses. In this proof-of-concept study, we developed four different PET probes that allow identification of species and strain specific bacteria as well as bacterial OMVs by targeting an exclusive metal transport system. We also demonstrated how these probes have the potential to be used for different durations depending on the type and severity of the pathologies being studied. The precise in vivo imaging by radiometal-labeled metallophores will be of great benefit not only to accurately diagnose bacterial infections, but also to facilitate the clinical translation of bacterial cancer therapy.

In typical embodiments of the invention, the PET probes are used as imaging tracers that detect bacterial infection within live tissues. For live tissue imaging, preferably the radiotracers of the invention can be administered to subjects in an amount suitable for in vivo imaging thereof, and to locate, diagnose, identify, evaluate, detect and/or quantify bacterial infection. Generally, a unit dosage comprising a PET probe radiotracer of the invention may vary depending on subject or patient considerations. Such considerations include for example, age, condition, sex, extent of disease, contraindications, or concomitant therapies.

The PET probe imaging compounds of the present invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes. Thus, the present compounds may be systemically administered, e.g., orally, in combination with a pharmaceutically acceptable vehicle such as an inert diluent or an edible carrier. They may be enclosed in hard or soft shell gelatin capsules, may be compressed into tablets, or may be incorporated directly with the food of the patient's diet. For oral administration, the PET probes disclosed herein may be combined with one or more excipients and used in the form of ingestible tablets, buccal tablets, troches, capsules, elixirs, suspensions, syrups, wafers, and the like.

Tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the probe, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the probe may be incorporated into sustained-release preparations and devices.

The probe may also be administered, for example, intravenously or intraperitoneally by infusion or injection. Solutions of the probe or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the probe in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Agents such as flavorings and additional antimicrobial agents can be added to optimize the properties for a given use.

Administration of a PET probe compositions of the invention to a subject may be local or systemic and accomplished orally, intradermally, intramuscularly, subcutaneously, intravenously, intra-arterially or intrathecally (by spinal fluid); or via powders, ointments, drops or as a buccal or nasal spray. A typical composition for administration can comprise a pharmaceutically acceptable carrier for the compound or radiotracer of the invention. Pharmaceutically acceptable carrier include, without limitation, aqueous solutions, non-toxic excipients comprising salts, preservative or buffers, amongst others known within the art.

The following examples provide illustrative methods and materials that can be used with embodiments of the invention.

### EXAMPLES

### Materials and Methods

### Chemicals

All reagents were purchased from commercial sources as analytical grade and used without further purification. Staphylopine (StP) was purchased from Toronto Research Chemicals Inc. (Toronto, Canada) and Yersiniabactin (YbT) from EMC Microcollections GmbH (Tuebingen, Germany). For animal studies, ⁶⁴Cu and ⁸⁹Zr were obtained from Mallinckrodt Institute of Radiology, Washington University School of Medicine.

### Bacterial Strains and Cell Culture Conditions.

Clinical isolates of pathogenic E. coli UTI89, E. coli O157:H7, K. pneumoniae, P. aeruginosa, S. aureus, nonpathogenic E. coli K12 (MG1655) and luciferase encoded E. coli Nissle were used in this study. Strains were grown in LB agar (BD Difco™) and LB medium (BD Difco™) as appropriate. The murine mammary carcinoma cell line 4T1 (ATCC CRL-2539) was cultured in RPMI containing 10% FCS. The cells were maintained at 37°° C. with 5% CO₂ in air, and subcultured twice weekly.

### Metallophore Production and Identification in Culture

E. coli UTI89 and S. aureus were grown for 20 hrs at 37° C. while shaking at 150 rpm. Subsequently, the cultures were centrifuged, and supernatants collected for metallophore identification via Liquid chromatography-mass spectrometry (LC-MS). Analyses were conducted using an Agilent 1290 LC-6540 Q-TOF system operated in positive ion mode using an AJS ESI ion source. 5 μL of the samples were injected into an Agilent Eclipse XDB-C18 column (3.5 um, 2.1×100 mm) with a flow rate of 0.5 ml/min and the following solvents: solvent A (0.1% (v/v) formic acid) and solvent B (0.1% (v/v) formic acid in acetonitrile). The gradient program was: 0-7 mins 5-95% B, 7-8 mins 95% B, 8-9 mins 95-5% B, and 9-10 mins 5% B.

### Radiometal-Metallophore Complexation and Stability

⁵⁵Co, ⁶⁸Ga and ⁸⁹Zr were produced at the University of Alabama. Complexation was performed by combining 50 μCi of radiometal with 5 or 10 μg of metallophore in 50 μl of various buffered media. Samples are incubated at 37° C. or 90°° C. for 1 hr and 30 mins respectively. Binding efficiency was determined via iTLC with 50 mM DTPA as the development buffer. The stability of ⁸⁹Zr labeled StP and YbT were assessed at various time points by incubating the probes in saline (0.9% NaCl) and in DTPA-supplemented saline.

### Radiolabeling for PET/CT Imaging

1-2 mCi of ⁸⁹Zr was mixed with 50 nmol of StP or YbT in 1M ammonium acetate (NH₄OAc) buffer solution (pH 7) at 90° C. for 30 mins. ⁶⁴Cu probes were prepared in the same conditions but at 37° C. for 1 hr. Since labeling efficiency for both the metals were >95% , the probes were administered without further purifications. Exosomes and OMVs were radiolabeled by incubating 1.2 mL of each of the nanovesicles with 1 mCi of ⁶⁴Cu-YbT. Unlabeled radiometal complexes were removed by separating the vesicles with PD-10 columns (GE Healthcare Life Sciences™).

### Animal Models

6-8 weeks old BALB/c mice were used for all experiments. For infection studies, 5×10⁶ cfu of bacteria were intramuscularly injected 6 hrs before probe administration. For each mouse, the right thigh was injected with the experimental bacteria and the left thigh with the control bacteria. Mice were injected with live and heat-killed (90° C. for 30 min) versions of the experimental bacteria as well. No more than two different bacteria were injected per mouse. For bacteria-based tumor imaging studies, 2.5×10⁶ 4T1 cells were subcutaneously injected and allowed to develop into a noticeable tumor (size >200 mm³) on the right flank of the mice. This was followed by a single intravenous (i.v.) administration of E. coli Nissle (5×10⁶ cfu) 3 days before probe PET probe administration. Radiolabeled nanovesicles were injected immediately after the tumor reached the appropriate size. All animal experiments were performed under anesthesia (2% isoflurane) by following protocols approved by the University of Cincinnati Biosafety, Radiation Safety, and Animal Care and Use Committees.

### Imaging and Ex Vivo Biodistribution Studies

200 μCi of PET probes were injected i.v. to selectively image bacteria in both infection and tumor models. 40 μCi of radiolabeled vesicles injected i.v. to study their tumor localization.

After the imaging studies, the mice were euthanized via carbon dioxide inhalation and cervical dislocation. Organs and tissues of interest were removed and weighed. Residual radioactivity in the samples was measured with a gamma counter and results expressed as percentage of injected dose per gram of organ (% ID/g).

### Statistical Analyses

All statistical analyses were carried out using the GraphPad Prism 7 software. Two-tailed unpaired student's t-test was performed to compare the means between two groups, whereas ANOVA was used to compare the means among three or more groups. Values of p<0.05 was considered statistically significant.

### Radiolabeling Procedure

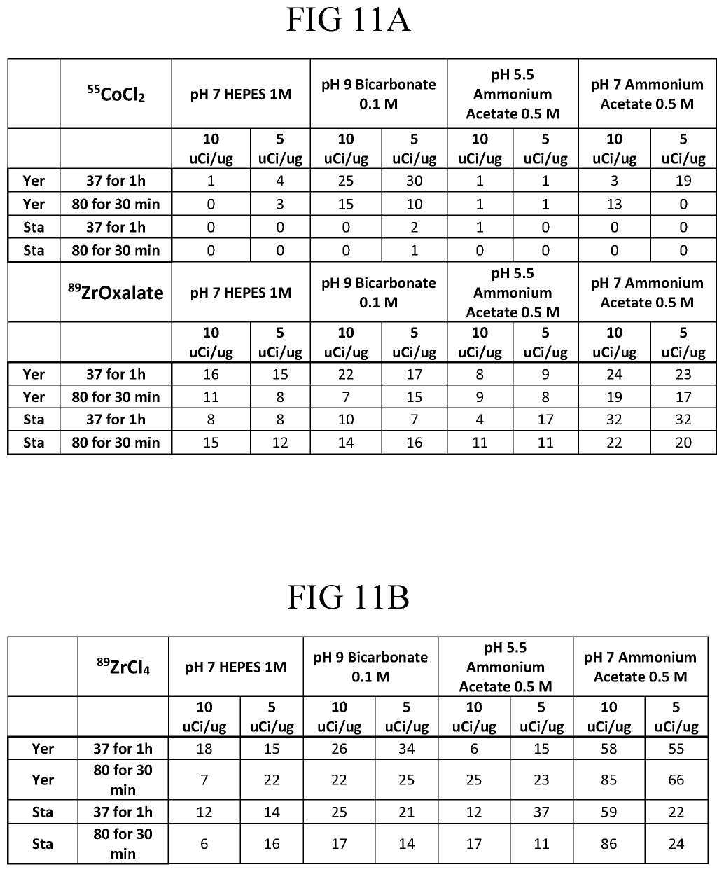

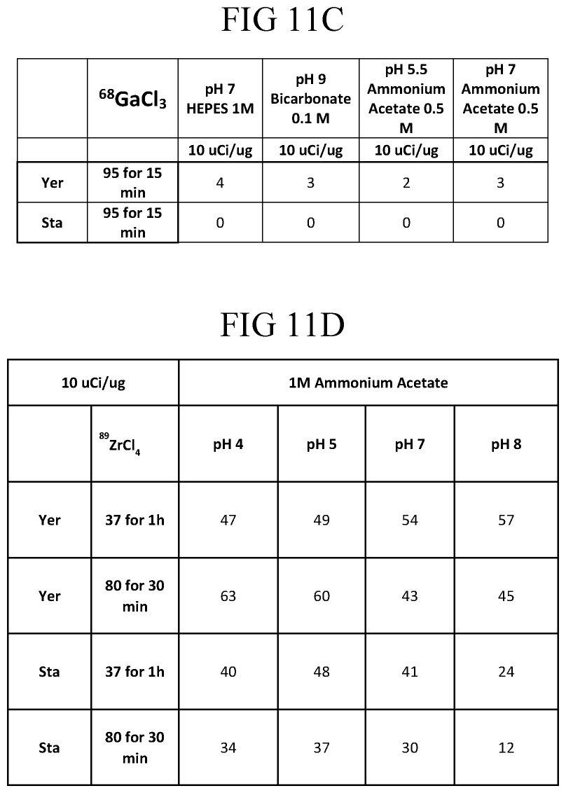

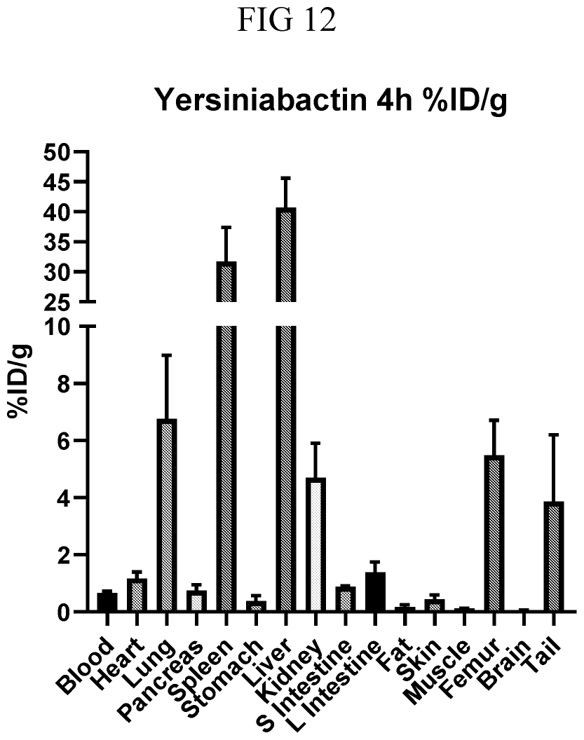

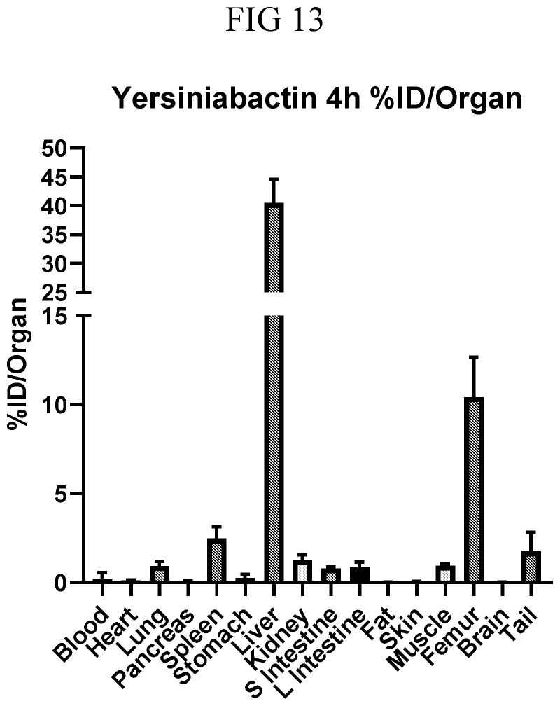

Buffers were prepared with trace metals grade reagents when available. For radiolabeling, 50 ul of buffer was combined with 50 ul of radiometal (2-10 ul). Either 5 ug or 10 ug of metallophore was added to give a final ratio of 10 uCi/ug or 5 uCi/ug respectively. Reactions were vortexed and centrifuged to combine. Then they were incubated at 37° C. on a thermomixer for 1 h. A 1 ul sample was removed for and spotted on an iTLC plate. The sample was returned to the thermomixer and incubated at 80° C. for 30 more minutes. Again, 1 ul was removed for TLC. TLC samples were developed in 50 mM DTPA. Radiolabeled metallophore remained at the baseline, while free radiometal traveled to the solvent front. Results are shown in FIGS. 11A-C.

### Example 1

Intramuscular myositis infection models involving E. coli UTI89, E. coli K12, K. pneumoniae, P. aeruginosa and S. aureus were developed. Furthermore, tumor models were developed via subcutaneous injection of 4T1 cells, followed by tail-vein injection of E. coli Nissle to study localization of bacteria in tumors. For probe preparation, 3 mCi of ⁶⁴Cu was added to 5 nmol of Ybt in 0.1 M ammonium acetate (pH 7) buffer. The reaction mixture was incubated on a mixer with 800 r.p.m. agitation at 37° C. for 1 hr. 200 μCi of the probe was injected intravenously per mouse and static PET/CT images were acquired 2 hr (for K. pneumoniae) and 24 hr (for E. coli Nissle and UTI89) post injection for imaging. E. coli K12, P. aeruginosa and S. aureus were the control bacteria. 200 μCi of ¹⁸F-FDG was used as the control probe. After the imaging studies, the mice were euthanized via carbon dioxide inhalation and cervical dislocation. Organs and tissues of interest were removed and weighed. Residual radioactivity in the samples was measured with a gamma counter. Results were expressed as percentage of injected dose per gram of organ (% ID/g).

### Example 2

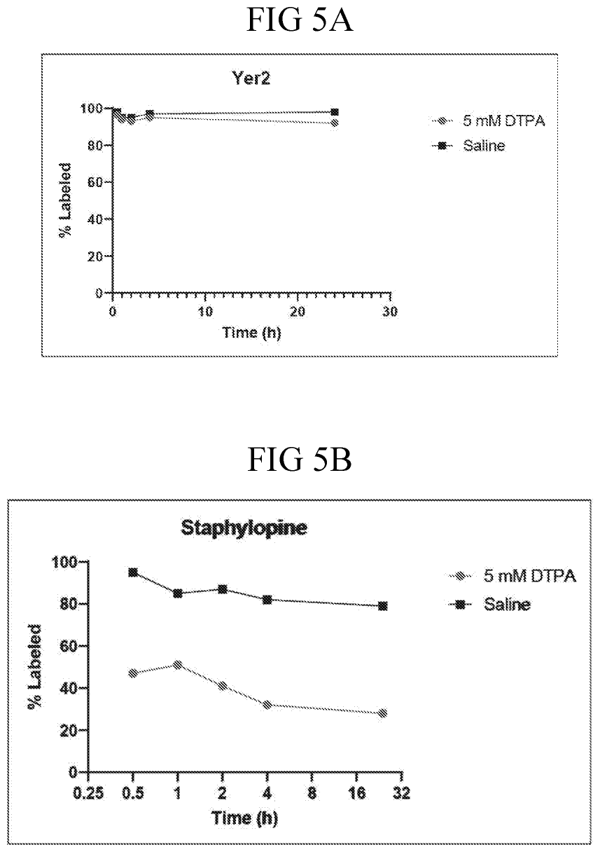

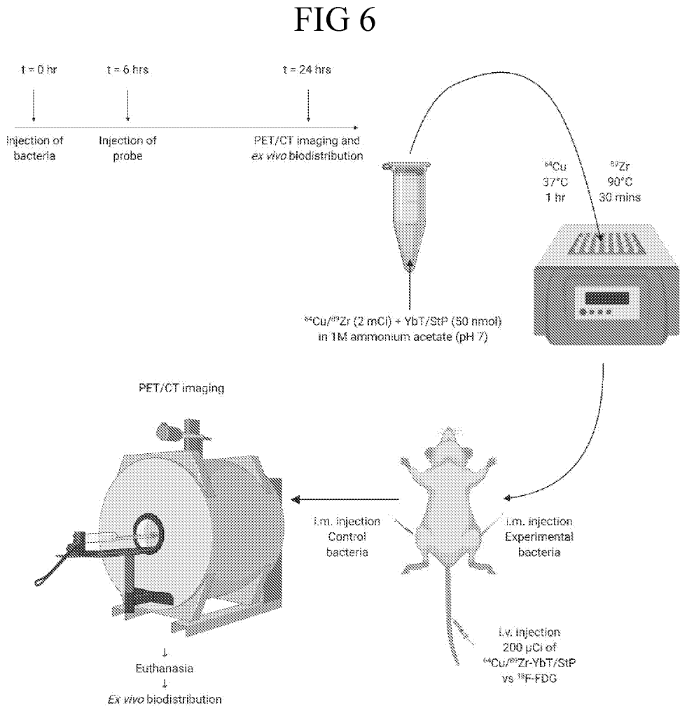

The stability of ⁸⁹Zr labeled StP and YbT was assessed. While ⁸⁹Zr remained bound to YbT in both saline and in diethylenetriaminepentaacetic acid (DTPA, a metal chelating agent) supplemented saline for 24 hrs in vitro (FIG. 5A), the radiometal dissociated significantly from StP within the first hour in the same conditions (FIG. 5B). Furthermore, ⁶⁴Cu-StP was observed mostly in the kidneys and bladder in mice, unlike ⁶⁴Cu and ⁸⁹Zr-labeled YbT probes that accumulated significantly in the liver than anywhere else in the body. Without being bound by theory, the rapid clearance of StP probes might be attributed to the hydrophilic nature (FIG. 2A) or dissociation of the radiometal from the molecule. On the other hand, the prolonged presence of the relatively lipophilic (FIG. 2B) YbT probes in the body confirms in vivo stability.

### Example 3

A test was conducted to determine whether the YbT probes could selectively differentiate its target, E. coli UTI89 (Gram-negative), from representative Gram-negative and Gram-positive bacteria that lack the FyuA receptor in vivo. P. aeruginosa (Gram-negative) and S. aureus (Gram-positive) were selected as the control bacteria. Tail-vein injections of ⁶⁴Cu-YbT in myositis infection models revealed significantly higher accumulation of the probe in UTI89 infected thigh muscles than in S. aureus or P. aeruginosa infected muscles.

### Example 4

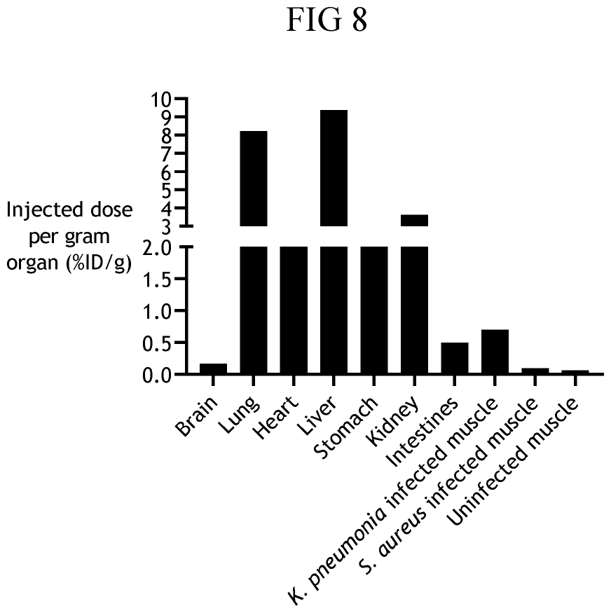

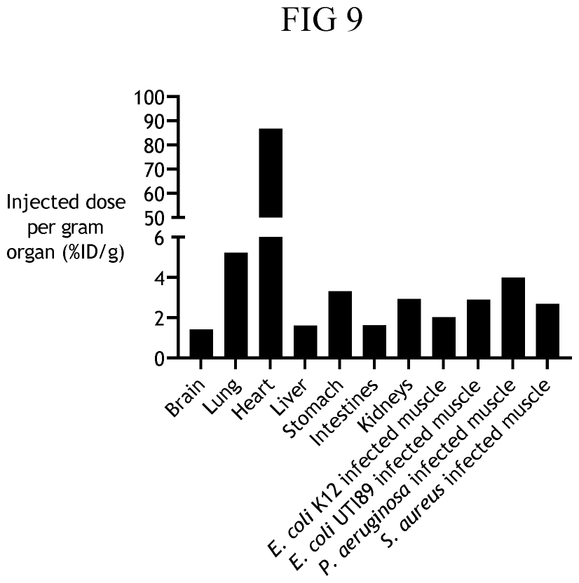

⁶⁴Cu-YbT was injected in mice that were intramuscularly infected with K. pneumoniae and S. aureus. PET/CT imaging and ex vivo biodistribution analysis yielded significantly higher radioactive signals from K. pneumoniae infected muscles compared to the control (FIG. 8)

### Example 5

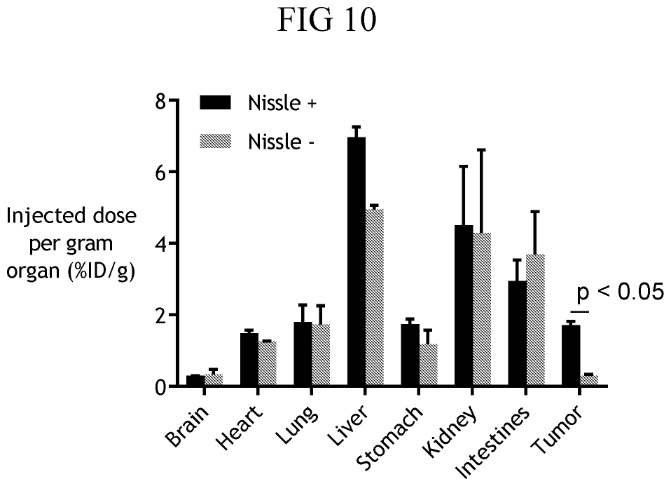

The specificity of the probes of the present invention was compared with the clinically available PET probe, ¹⁸F-FDG, and found that the latter was unable to distinguish any of the pathogenic bacteria we used in our experiments (FIG. 10). It is well-known that ¹⁸F-FDG is taken up by cells that express high levels of glucose transporters (e.g. neutrophils, macrophages and activated leukocytes) that are involved in both septic and aseptic inflammation. Without being bound by theory, we believe that ¹⁸F-FDG accumulated inside the white blood cells that infiltrated the thigh muscles due to presence of infectious bacteria. The lack of a substantial signal from the muscle injected with the non-pathogenic commensal E. coli K12 indicates that the bacteria was not able to cause an inflammatory response itself.

### Example 6

Subcutaneous tumor models were developed by injecting the metastatic 4T1 cancer cells in syngeneic mice. When the tumors developed into a noticeable size (˜200 mm³), intravenous injection of Nissle that encode luciferase genes was performed. Usually, more than 99% of the administered bacteria get cleared from the animals, leaving only a small percentage colonize the tumor. Hence, 3 days were allowed for the bacteria to localize and proliferate in the hypoxic core of the tumor before administering the PET probes in the mice. The control mice were not administered with Nissle. ⁶⁴Cu-YbT was observed to specifically accumulate in tumors of Nissle administered mice (FIG. 10), thus allowing bacteria-specific cancer imaging.

### Results

PET/CT imaging revealed significantly more pronounced PET signals from thigh muscles infected with UTI89 and K. pneumoniae than from those with the control bacteria. ¹⁸F-FDG accumulated in all infected thigh muscles non-specifically. A substantially higher PET signal was also observed in the tumor of the mouse that was injected with E. coli Nissle than from that without the bacterial injection. Ex vivo biodistribution studies revealed concordant results. E. coli Nissle, UTI89, and K. pneumoniae possess the transcription machinery to synthesize and secrete Ybt, and the FyuA receptor to internalize metal-Ybt complexes (⁶⁴Cu-Ybt in this instance) compared to E. coli K12, P. aeruginosa or S. aureus, which utilize different mechanisms for trace metal uptake.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A probe for positron emission tomography (PET) selected from the group consisting of:
yersiniabactin (Ybt) labeled with Copper-64;
staphylopine (StP) labeled with Copper-64;
yersiniabactin (Ybt) labeled with Zirconium-89; and
staphylopine (StP) labeled with Zirconium-89.

2. The probe of claim 1 wherein the probe is yersiniabactin (Ybt) labeled with Copper-64.

3. The probe of claim 1 wherein the probe is staphylopine (StP) labeled with Copper-64.

4. The probe of claim 1 wherein the probe is yersiniabactin (Ybt) labeled with Zirconium-89.

5. The probe of claim 1 wherein the probe is staphylopine (StP) labeled with Zirconium-89.

6. A method of identifying a bacterial infection in vivo in a mammal, the method comprising the steps of:
(a) administering a composition to the mammal comprising a positron emission tomography (PET) probe selected from the group consisting of:
yersiniabactin (Ybt) labeled with Copper-64;
staphylopine (StP) labeled with Copper-64;
yersiniabactin (Ybt) labeled with Zirconium-89; and
staphylopine (StP) labeled with Zirconium-89;
(b) allowing the probe to accumulate in the mammal; and
(c) observing the accumulated probe in the mammal using a positron emission tomography process;
so that a concentration of the probe in the mammal is observed.

7. The method of claim 6, wherein the bacterial infection that is observed in vivo comprises bacteria selected from the group consisting of E. coli UTI89 (Gram-negative) and K. pneumonia.

8. The method of claim 6 wherein the bacterial infection that is observed in vivo comprises E. coli UTI89 (Gram-negative) bacteria.

9. The method of claim 6, wherein the bacterial infection that is observed in vivo comprises K. pneumonia bacteria.

10. The method of claim 6, wherein the positron emission tomography (PET) probe is yersiniabactin (Ybt) labeled with Copper-64.

11. The method of claim 6, wherein the positron emission tomography (PET) probe is yersiniabactin (Ybt) labeled with Zirconium-89.

12. The method of claim 6 wherein the concentration of the probe is used to detect a urinary tract infection.

13. The method of claim 6 wherein the concentration of the probe is used to monitor treatment response, progression, or both in antibiotic resistant E. coli.

14. The method of claim 6, wherein the positron emission tomography probe is administered to the mammal in combination with a pharmaceutically acceptable compound comprising a diluent, a carrier, or a binding agent.

15. A method of selectively observing therapeutic bacteria in vivo in a mammal, the method comprising the steps of:
(a) administering a composition to the mammal comprising therapeutic bacteria;
(b) allowing the therapeutic bacteria to selectively accumulate in the mammal;
(c) administering a composition to the mammal comprising a positron emission tomography (PET) probe selected from the group consisting of:
yersiniabactin (Ybt) labeled with Copper-64;
staphylopine (StP) labeled with Copper-64;
yersiniabactin (Ybt) labeled with Zirconium-89; and
staphylopine (StP) labeled with Zirconium-89;
(d) allowing the probe to selectively accumulate in the mammal; and
(e) observing the accumulated probe in the mammal using a positron emission tomography;
so that the therapeutic bacteria is selectively observed in vivo in the mammal.

16. The method of claim 15, wherein the therapeutic bacteria is a non-pathogenic strain of E. coli.

17. The method of claim 15, wherein the positron emission tomography probe is administered to the mammal in combination with a pharmaceutically acceptable compound comprising a diluent, a carrier, or a binding agent.

18. The method of claim 15, wherein the positron emission tomography (PET) probe is yersiniabactin (Ybt) labeled with Copper-64.

19. The method of claim 15, wherein the positron emission tomography (PET) probe is yersiniabactin (Ybt) labeled with Zirconium-89.
